SEQUENCE LISTING (3 sequences)

SEQ ID NO: 1 - DNA, 22 bp, artificial sequences
  primer A
tcgtaagcgttctgacgttatc

SEQ ID NO: 2 - DNA, 24 bp, artificial sequences
  primer B
catgatgccgtcagaggcttcgag

SEQ ID NO: 3 - DNA, 23 bp, artificial sequences
  probe
acctgaaagcgcacggcggcgaa

(19) United States
(12) Patent Application Publication
(10) Pub. No.: US 2014/0134718 A1
(43) Pub. Date: May 15, 2014

(54) FILTER MODULE IN BIOMOLECULE ISOLATION

(71) Applicants: Margit Hiesinger, Wuppertal (DE); Karin Schulte, Dusseldorf (DE)

(72) Inventors: Margit Hiesinger, Wuppertal (DE); Karin Schulte, Dusseldorf (DE)

(73) Assignee: QIAGEN GmbH, Hilden (DE)

(21) Appl. No.: 14/130,443

(22) PCT Filed: Jun. 29, 2012
(86) PCT No.: PCT/EP2012/002759

§ 371 Date: Dec. 31, 2013

(30) Foreign Application Priority Data
Jul. 1, 2011 (EP) 1105418.6

(51) Int. Cl. C12N 15/10

(52) U.S. Cl. CPC C12N 15/1017
USPC 435/309.1; 422/535; 536/25.4

(57) ABSTRACT

The present invention relates to a device for rapid isolation of target molecules from cell lysates and other liquid mixtures comprising particulate material, a method for isolating the target molecules, in particular nucleic acids, using said device and a kit for carrying out said method comprising said device.

The present invention relates to a device for rapid isolation of target molecules from cell lysates and other liquid mixtures comprising particulate material, a method for isolating the target molecules, in particular nucleic acids, using said device and a kit for carrying out said method comprising said device.

In biochemical and biological procedures, it is often desirable to isolate and collect a particular type of molecules from a liquid mixture or sample comprising particulate or flocculated material. This can be for example achieved by filtering the mixture resulting in retaining the solid or flocculated material on/in the filter material. The flow-through can be contacted with a material that selectively binds the desired molecules resulting in capturing the desired molecules from the filtered liquid. The desired molecules may then be eluted from the binding material and collected.

For example, plasmid purification from bacteria typically involves the generation of a cell lysate containing soluble plasmid material and insoluble cell debris, protein and genomic DNA particles.

The lysate is clarified by removing the insoluble particulate material, typically by centrifugation or by a filtration device. The clarified lysate is then transferred to a nucleic acid binding solid material which binds the desired plasmid DNA. After optional washing steps the target DNA is then eluted from the binding material and collected.

Clarification of cell lysates and elution of plasmid DNA are widely used and described in the art, wherein the filtering step in numerous different embodiments is carried out by a filter device in combination with a DNA binding column. Such solutions are described e.g. in WO 95/02049 A1, KR 2004/085927 A, DE 202005010007 U1, WO 2008/121121 A2, WO 2008/150838 A1, WO 2009/157679 A1 or WO 2010/075116 A2.

Further solutions are described where in one column a filter for precipitate retention and a DNA binding material is contained, thus the filter cannot be removed. Such an embodiment is shown e.g. in WO 2008/150826 A1.

In several of the prior art devices two different types of filters for retaining the solids, particulates or flocculants are contained, i.e. a pre-filter and a depth filter. The pre-filter is commonly a coarse filter retaining e.g. non-lysed cells, cell debris and further particles, wherein the depth filter retains the finer precipitate or flocculants. The pre-filters are commonly made of a rigid material like sintered porous polyethylene or polypropylene (e.g. in WO 2008/50838 A1, WO 2008/50826 A1, WO 2005/12521 A1 or US2006252142 A), glass (WO2009/58414 A1), a metal mesh (WO 2003/46178A1) or zeolith (WO 2009/60847 A1). The depth filter is most commonly prepared from fiber materials in form of membranes or layers, including paper, wherein the materials are selected from polysaccharides like cellulose, cellulose acetate, plastics like polyethylene, polypropylene, Teflon (PTFE), polyacrylate, polyamide or polyvinylidenefluoride or polymers including sulfone groups like polyphenylsulfone, polyethersulfone, polysulfone, polyarylsulfone or polyphenylsulfone.

Further materials described for retention of the solids, precipitates and flocculates are beads or meshes of glass, metal or plastics.

In two prior art documents the use of hydrogel columns for retention of debris and precipitates are discussed (WO 2009/157679 A1 and WO 2009/157680 A1). The used material is agarose, allowing the nucleic acids to pass but retaining most of the contaminants of the lysate.

The cell lysate includes genomic DNA representing a high molecular weight biomolecule which is very sensitive to shear forces. Most of the filter materials used in the prior art are quite rigid. Thus, if high forces are applied to the genomic DNA, as for example during high speed centrifugation, such rigid material results in shearing the genomic DNA. The fragments of genomic DNA can then pass the filters and are bound to the DNA binding matrix resulting in a contamination of the desired plasmid DNA.

On the other hand, when agarose or another hydrogel is used as filter material, the material itself is very sensitive to external forces. As discussed in WO 2009/157680 A1 the column containing agarose as filter has to be prepared shortly before use, but then the column can be spinned in a centrifuge only in the range between 1000 rpm and 3000 rpm. If lower rpm is used, the DNA doesn't run through the agarose column, if higher rpm is used the agarose is damaged.

Therefore, it was an object of the present invention to develop a method and a device for clarifying a liquid mixture comprising at least one type of molecule which is sensitive to shear forces, followed by selectively binding a desired molecule of the mixture and collecting the bound material, wherein the contamination of the desired molecule is minimized.

This object is met by a clarification/binding device comprising a filter module, wherein said filter module comprises an elastic filter material, the use of said device in a target isolation method and a kit for target isolation containing such a device.

A clarification/binding device according to the present invention represents a device which is able to clarify a suspension comprising a liquid phase and solid particles, precipitates and/or flocculates by separating and/or retaining the particles, precipitates and/or flocculates from the liquid phase, e.g. by filtration, and further is able to bind at least one target molecule of the ingredients of the liquid phase passed through the clarification means (e.g. the filter), thereby separating said target from the remaining liquid phase.

In a preferred embodiment the invention provides a clarification/binding device for isolation of at least one target molecule from a sample comprising a filter module and a target binding column. The clarification/binding device can be a single column clarification/binding device, wherein the filter module is contained in a column further comprising a target binding material. Preferably the filter module is removable from said column. In a further embodiment the clarification/binding device is a dual column clarification/binding device, wherein the filter module is in form of a further column which is inserted in the binding column. The filter module in form of a column containing the filter material preferably is configured to receive the lysate.

The filter module comprises at least one filter material which is a “deep bed filtration” material essentially avoiding blockage of the filter. Said material is preferably elastic, which means that it is deformable e.g. by hand, preferably it is soft and flexible. The material preferably is selected form any foam (foamed material) or sponge having open pores (open cells). Such materials are exemplified by foamed polyethylene, polypropylene, polyurethane (including polyester-based polyurethane), polyester, polyether, polystyrene, melamine, or other plastic polymers having open cells or by natural sponges like Porifera, animal fiber sponge or plant fiber sponge. Said materials are known in the art, for example for air filter, e.g. for medical instruments, cushions for bandages, cleaning use or similar.

The pores of the elastic material, preferably the foam or sponge, should be preferably in the range from 10 μm to 1000 μm, more preferred in the range from 25 to 500 μm and most preferred in the range from 50 to 200 μm. The foam has preferably a cell number per centimeter of 5 to 50, preferably from 20 to 40 cells/cm.

The foam or sponge preferably is a “self-supporting” foam or sponge. This means that the foam or sponge is an elastic material, deformable by pressure, however, returning essentially to its original shape when the applied pressure is removed. In a particularly preferred embodiment the elastic material is a soft material, which may be easily deformed by the power of a human finger.

This elasticity as well can be determined by the so-called compression hardness (or compression strenth of the foam), e.g. as measured by DIN EN ISO 3386. It can be determined by compressing a standard sized piece of the foam to a predetermined amount (commonly to 40%) and measure the force required to obtain that compression in kPa or N/m². The compression hardness (to 40%) of the preferred materials is in the range of 0.5 to 50 kPa, preferably in the range of 1 to 30 kPa, more preferred from 1 to 20 kPa and particularly preferred from 2 to 10 kPa.

Due to the elastic and flexible characteristics of the used filter material shearing of a shear sensitive molecule like e.g. genomic DNA is reduced and the amount of genomic DNA fragments in the eluate is very low, comparable to methods without the use of a filter, like e.g. centrifugation.

The filter module further preferably comprises one or two layers of a second filter, preferably below the elastic filter. Preferably the second filter as well should not be prepared from a rigid material shearing sensitive molecules like the genomic DNA during high speed centrifugation. Said second filter can represent a commonly used depth filter, preferably prepared from fiber material. Said second filter may be in form of a membrane or layer, wherein the material may be selected from polysaccharides like cellulose, including paper, cellulose acetate, plastics like polyethylene, polypropylene, Teflon® (PTFE), polyethyleneterephthalat (PET), polyacrylate, polyamide, in particular Nylon®, polyvinylidenefluoride, polymers including sulfone groups like polyphenylsulfone, polyethersulfone, polysulfone, polyarylsulfone or polyphenylsulfone or not DNA binding silica.

The pores of the second filter are smaller that the pores of the elastic filter and are preferably in the range of 0.1 to 50 μm, preferably in the range of 0.5 to 30 μm, most preferred in the range of 1 to 10 μm. As far as the pore sizes of the elastic filter and the second filter described here are overlapping in its described ranges it should be clear that the pore size of the second filter should be smaller than the pore size of the elastic filter.

The thickness of the elastic filter and the thickness of the second filter(s) is not limiting the present invention, however, it is preferred that the thickness of the elastic filter is for example between 1 and 10 mm, preferably 2 to 8 mm, more preferred 3 to 5 mm. The thickness of the second filter(s) is preferably lower than the thickness of the elastic filter, since intentionally the second filter(s) retain(s) less material than the elastic filter. Accordingly the thickness of the second filter(s) may range e.g. from 0.1 to 1 mm each.

The filter(s) allow(s) the liquid phase to pass and retain(s) essentially all of the solid/particulate/flocculated material. The filter(s) is/are mounted in the filter module in a way that the lysate has to pass the filter(s) and is not allowed to by-pass. Accordingly, e.g. if a filter column is used, the filter(s) has/have contact to the side walls of said column or is/are placed in a holder having contact with said side walls. For example the elastic filter can be sized to fit inside of the column with contact to the side walls of the column, in particular when it is wet, e.g. by “oversizing” the filter compared to the inner diameter of the column. In this case the filter “grips” inside of the column. Further the filter can be hold in place by a ring under and/or over said filter. If a ring is placed over the filter this has the additional advantage that the liquid lysate is directed through the elastic filter and is hindered to by-pass the filter by running down the walls of the column.

To provide the filter module of the present invention the elastic filter, e.g. the foam or sponge, is placed inside of a container having an inlet and an outlet like e.g. a tube, a column, a syringe or similar. Said container can be made of plastic, metal, composite material, glass, or any combination thereof, of any other suitable non-reactive or biocompatible material. The container can be fabricated using an injection moldable material that is able to withstand the force created by a centrifuge or moderate vacuum pressure. If desired, a second filter as well is placed inside said container, preferably below the elastic filter. At least one of the filters may be supported inside of the container e.g. by designing the container in a way that the bottom end represents a liquid permeable supporting structure like e.g. a mesh, a fence, or any similar supporting structure like crossing, cartwheel or similar, or a channels including face or the filters are supported inside of the container by a supporting means, e.g. a holder, a ring, a mesh, a fence, or any similar supporting structure like crossing, cartwheel or similar, or a channels including face, or it may be adhered to the walls of the container, e.g. by a glue, gum, seal or similar adhesive. Alternatively, the support element can be a modification of the inner surface of the binding column, such as an annular ridge formed on the inner surface of the internal bore. In a preferred embodiment a second filter is placed below of a foam or sponge into a column, wherein the second filter may be supported. The second filter itself can then serve as a support for the elastic filter. The so prepared filter module in form of a column can be inserted into a further column having therein a binding material for the desired target, resulting in a dual column device.

The binding column preferably is configured to receive the filtered sample from the filter module. The binding column comprises a binding material for binding at least one target molecule. Said binding column may be represented by any of the columns known in the art for binding desired targets, in particular nucleic acids like plasmid DNA or RNA or proteins passed through the elastic filter or the filter module. Unless defined otherwise, all technical and scientific terms used herein have the same meaning as commonly understood by one of ordinary skill in the art to which this invention belongs. Generally, the nomenclature used herein are well known and commonly employed in the art. Terms of orientation such as “up” and “down”, “top” and “bottom”, “above” and “below” or “upper” or “lower” and the like refer to orientation of parts during use of a device. Where a term is provided in the singular, the plural of that term is also contemplated.

The term “mini preparation” or “mini prep” as used herein refers to a scale of purification from a starting culture volume of approximately 0.5-5 ml. Columns and other devices used in mini prep purification can also range from approximately 0.5 ml to approximately 5 ml. The term “midi/maxi preparation” or “midi/maxi prep” refers to a scale of purification starting from a culture volume of 5-100 ml. Columns and other devices used in midi prep purification can range from approximately 5 ml to approximately 15 ml or from approximately 5 ml to approximately 25 ml, and columns and other devices used in maxi prep purification can range from approximately 25 ml to approximately 100 ml.

The term “column” and “columns” as used herein refers to a device or container having an inlet and an outlet and which are able to hold a liquid. While columns generally refer to devices and containers having approximately cylindrical shapes, it is understood that the term “column” as used herein can refer to devices or containers having any shape, in particular a conical shape, or others, including but not limited to predominantly spherical, pyramidal, rectangular, irregular shapes and combinations thereof.

The term “target biomolecule” or “target molecule” may comprise nucleic acids, proteins, lipids, glycolipids, pathway products or sugars, wherein the preferred target molecules are nucleic acids or proteins, particularly preferred nucleic acids.

The term “protein” or “proteins” as used herein include full length proteins, protein fragments, proteins in their native state or denatured proteins. Mixture of proteins can be a mixture of full length proteins, a mixture of protein fragments, or a mixture of full length proteins and protein fragments.

The term “nucleic acid” as used herein includes both DNA and RNA without regard to molecular weight or source. Nucleic acids include the full range of polymers of single or double stranded nucleotides, including chemically modified nucleotides, as known in the art that are capable of forming base pairs, joinable with other nucleic acids, and cleavable by endo- or exonucleases. Nucleic acids may be derived from any natural source or may be modified. A DNA molecule is any DNA molecule of any size, from any source, including DNA from viral, prokaryotic and eukaryotic organisms, as well as synthetic DNA and variants, derivatives and analogs thereof. The DNA may be genomic DNA or extrachromosomal DNA. A RNA molecule is any RNA molecule of any size, from any source, including RNA from viral, prokaryotic and eukaryotic organisms, as well as synthetic RNA and variants, derivatives and analogs thereof. The RNA and DNA may be single stranded or double stranded, linear or circular, or supercoiled. The preferred nucleic acids are designated “target nucleic acids”.

In accordance with the invention, “target nucleic acids,” preferably includes extrachromosomal DNA, e.g., plasmids and their fragments, vectors and their fragments, phagemids, cosmids, BACs, PACs, YACs, mitochondrial nucleic acid molecules, chloroplast nucleic acid molecules, or combinations thereof. In particular, any vector and/or plasmid is preferred. They may be either commercially available, or synthesized, or engineered, or derived thereof. Such vectors and/or plasmids may be used for or derived from cloning or subcloning nucleic acid molecules of interest and therefore recombinant vectors containing inserts, nucleic acid fragments or genes may also be isolated in accordance with the invention. General classes of vectors of particular interest include prokaryotic and/or eukaryotic cloning vectors, expression vectors, fusion vectors, two-hybrid or reverse two-hybrid vectors, shuttle vectors for use in different hosts, mutagenesis vectors, transcription vectors, short hairpin vectors, vectors for receiving large inserts (yeast artificial chromosomes (YACs), bacterial artificial chromosomes (BACs) and P1 artificial chromosomes (PACs)) and the like. Other vectors of interest include viral origin vectors (M13 vectors, bacterial phage vectors, baculovirus vectors, adenovirus vectors, and retrovirus vectors), high, low and adjustable copy number vectors, vectors which have compatible replicons for use in combination in a single host (e.g., pACYC184 and pBR322) and eukaryotic episomal replication vectors (e.g., pCDM8). The vectors contemplated by the invention include vectors containing inserted or additional nucleic acid fragments or sequences (e.g., recombinant vectors) as well as derivatives or variants of any of the vectors described herein. Expression vectors useful in accordance with the present invention include chromosomal-, episomal- and virus-derived vectors, e.g., vectors derived from bacterial plasmids or bacteriophages, and vectors derived from combinations thereof, such as cosmids and phagemids.

Any cell, tissue, or organism may be used as the source of the target molecules to be isolated, such that the target molecules that are contained in the cell, tissue, or biological source (or portion thereof) are released from the cell, tissue, or organism. A cell may be prokaryotic or eukaryotic. An organism may be prokariotic, eukaryotic or viral, etc., and generally refers to any cell that contains a target molecule, e.g. a nucleic acid of interest. The terms “host” or “host cell” may be used interchangeably herein. For examples of such hosts, see Maniatis et al., “Molecular Cloning: A Laboratory Manual,” Cold Spring Harbor Laboratory, Cold Spring Harbor, N.Y. (1982). Preferred prokaryotic hosts include, but are not limited to, bacteria of the genus Escherichia (e.g., E. coli), Bacillus, Staphylococcus, Agrobacter (e.g., A. tumefaciens), Streptomyces, Pseudomonas, Salmonella, Serratia, Caryophanon, etc. The most preferred prokaryotic host is E. coli. Bacterial hosts of particular interest in the present invention include E. coli strains K12, DH10B, DH5alpha, HB101, JM109, XI1 blue, Top10, Top10F and QIAGEN EZ Preferred eukaryotic hosts include, but are not limited to, fungi, fish cells, yeast cells, plant cells and animal cells, particularly insect cells, and mammalian cells including human cells, CHO cells, VERO cells, Bowes melanoma cells, HepG2 cells, and the like. Cells may be transformed cells, established cell lines, cancer cells, or normal cells. Exemplary animal cells are insect cells such as Drosophila cells, Spodoptera Sf9, Sf21 cells and Trichoplusa High-Five cells; nematode cells such as C. elegans cells; and mammalian cells such as COS cells, CHO cells, VERO cells, 293 cells, PERC6 cells, BHK cells and human cells. Any virus may also be used as a cellular source of biological macromolecules, particularly nucleic acid molecules, in accordance with the invention. Also suitable for use as sources of biological macromolecules are blood or mammalian tissues of organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus. These cells, tissues and organs may be normal, transformed, or established cell lines, or they may be pathological such as those involved in infectious diseases (caused by bacteria, fungi or yeast, viruses (including AIDS) or parasites), in genetic or biochemical pathologies (e.g., cystic fibrosis, hemophilia, Alzheimer's disease, schizophrenia, muscular dystrophy or multiple sclerosis), or in cancers and cancerous processes. Other cells, tissues, viruses, organs and organisms that will be familiar to one of ordinary skill in the art may also be used as sources of biological macromolecules for the preparation of biological macromolecules according to the present invention. In accordance with the invention, a host or host cell may serve as the cellular source for the desired macromolecule to be isolated.

As used herein, “cell disrupting” or “cell lysing” refers to cell opening using a composition or a component of a composition that effects lysis, rupture, or poration of the cells, tissues, or organisms used as the source of the biological macromolecules to be isolated, such that the macromolecules that are contained in the cell, tissue, or biological source (or portion thereof) are released from the cell, tissue, or organism. According to the invention, the cells, tissues, or organisms need not be completely lysed, ruptured or porated, and all of the macromolecules of interest contained in the source cells, tissues or organisms need not be released therefrom. Preferably, a cell disrupting or cell lysis compound or composition effects the release of at least 25 percent, 50 percent, 75 percent, 80 percent, 85 percent, 90 percent, 95 percent, 97 percent, 99 percent, or more of the total biological macromolecules of interest, that are contained in the cell, tissue, or organism.

In accordance with the invention, the cells may be lysed or disrupted by contacting them with a composition or compound which causes or aids in cell lysis or disruption, although mechanical or physical forces (e.g., pressure, sonication, temperature (heating, freezing), and/or freeze-thawing etc.) may be used in accordance with the invention. In addition, any combination of mechanical forces, physical forces or lysis compositions/compounds maybe used to disrupt/lyse the cells, so long as the method does not substantially damage the biological macromolecules of interest.

The cell disrupting or cell lysing compound or composition used is not limiting the invention. The components comprised in the lysing composition are preferably adapted to the desired target biomolecule to be isolated or purified. Which components are preferably included for what type of desired biomolecule is known to a skilled artisan and doesn't limit the present invention. It may comprise one or more detergents, such as sodium dodecylsulfate (SDS), Sarkosyl, Triton X-100, Tween 20, NP-40, Nalkylglucosides, N-alkylmaltosides, glucamides, digitonin, deoxycholate, 3-[(3cholamidopropyl)-dimethylammonio]-1-propane-sulfonate (CHAPS), cetyltrimethyl-ammoniumbromide (CTAB), or Brij 35. The concentration may be any suitable, e.g. about 0.01 percent −10 percent (w/v), more preferably about 0.1 percent −5 percent. One or more chaotropic agents such as sodium iodide, sodium perchlorate, guanidine or a salt thereof or urea may be present. Further, one or more enzymes may be present such as lysozyme, lyticase, zymolyase, neuraminidase, Novozym 234, streptolysin, cellulysin, mutanolysin or lysostaphin. One or more inorganic salts may be present such as sodium chloride, potassium chloride, magnesium chloride, lithium chloride, or praseodymium chloride, e.g. at a concentration of about 1 mM to 5M. One or more organic solvents such as toluene, phenol, butanol, isopropyl alcohol, isoamyl alcohol, ethanol, an ether (e.g., diethyl ether, dimethyl ether, or ethylmethyl ether), or chloroform may be present as far as they have no negative effects to any of the filters used according to the invention. Any other compound which disrupts the integrity of (i.e., lyses or causes the formation of pores in) the membrane and/or cell wall of the cellular source of biological macromolecules (e.g., polymixin B), may be present, or combinations of any of the foregoing. The compositions may also comprise other components, such as chelating agents (e.g., disodium ethylenediaminetetraacetic acid (Na EDTA), EGTA, CDTA), one or more proteases (Protinase K, Pronase, pepsin, trypsin, papain, subtilisin) or any combination of the foregoing. Desired concentrations and combinations of the active ingredients of the lysis/disruption compositions are not limiting the invention and may be readily determined by those skilled in the art with routine experimentation.

The term “clarification” as used herein refers to the process of removing unwanted solid, precipitated or flocculated materials like e.g. cellular debris and/or large insoluble molecules from a liquid mixture, e.g. a cell lysate. Common methods of clarifying a cell lysate include centrifugation and filtration. Preferably the unwanted debris is essentially retained in the filter module, allowing the essentially clarified lysate to pass through the binding column containing the binding material.

The term “elution” as used herein refers to the release of the desired target molecules bound by the binding matrix by means of a solvent. The terms “eluting solution”, “elution buffer” and “elution solution” refer to the solvent used to release a molecule from the material. The term “eluate” refers to the liquid solution resulting from an elution and containing the desired molecule.

The “eluate” may contain nucleic acid that may be considered “isolated”. As used herein, the term “isolated” (as in “isolated biological macromolecule”) means that the isolated material, component, or composition has been at least partially purified away from other materials, contaminants, and the like which are not part of the material, component, or composition that has been isolated. For example, an “isolated biological macromolecule” is a macromolecule that has been treated in such a way as to remove at least some of the other macromolecules and cellular components with which it may be associated in the cell, tissue, organ or organism. In particular, the phrases “isolated biological macromolecule,” “isolated nucleic acid molecule” or “isolated plasmid” refer to macromolecule preparations or plasmid preparations which contain about 10 percent, 20 percent, 30 percent, 40 percent, 50 percent, 55 percent, 60 percent, 65 percent, 70 percent, 75 percent, 80 percent, 85 percent, 90 percent, and 93 percent, preferably more than 95 percent, 97.5 percent, and 98 percent, and most preferably more than 99 percent, 99.5 percent, and 99.9 percent (percentages by weight) of the biological macromolecule of interest. As one of ordinary skill will appreciate, however, a solution comprising an isolated macromolecule may comprise further ingredients e.g. one or more buffer salts and/or a solvents, e.g., water or an organic solvent and the like, and yet the macromolecule may still be considered an “isolated” macromolecule with respect to its starting materials.

The nucleic acid molecules that are isolated by the devices, methods and kits of the present invention may be further characterized or manipulated, for example by amplification, cloning, sequencing, labeling, transfections, transformations, in vitro transcription, in vitro translation, nucleic acid synthesis, endonuclease digestion, other enzymatic modifications and the like, methods that are routinely used by one of ordinary skill in the art.

By the present invention devices and methods are provided suitable for clarifying or filtering a liquid mixture or sample comprising shear-sensitive macromolecules, followed by binding of a desired molecule from the clarified or filtered liquid or sample to a binding material. The isolation of the desired molecule can further comprise the subsequent elution of the desired molecule from the binding material. In a preferred embodiment the present invention provides a dual device comprising a filtering module in form of a clarification/filtering column inserted at least partially into a binding column, where the clarification column retains solid, precipitated or flocculated material from the liquid mixture and the binding column binds a desired target molecule or molecules from the filtered liquid. The bound target molecule can then be eluted from the binding material if desired.

In a further embodiment of the present invention the filter module is inserted in the same column as the binding material. In such an embodiment the filter module is not a column, but e.g. a disc, enclosing the elastic and optionally the second filter described above, which can be placed on an intended lace inside of the binding column. In such a case the binding column preferably is designed in a way that the filter module is hold in its intended place by a means described above. According to the present invention it is preferred that the filter module is removed after passing the sample through the filter. Accordingly the filter module preferably is removable from the binding column.

The clarification/binding device can be used for the clarification of a sample and the subsequent isolation of at least one target molecule from the sample, wherein the contamination of fragments of shear-sensitive molecules is minimized. The sample can be a liquid sample or a dry sample that has been suspended in a liquid. For example, the device described herein can clarify a cell lysate sample and isolate a nucleic acid molecule from the clarified lysate. After the sample, for example, lysate, is filtered and the target molecule(s) is captured by the binding material, the internal filter module (along with the unwanted debris) can be removed and discarded. The nucleic acid or other target molecule(s) bound to the binding material can then be optionally washed and eluted.

In the above dual column embodiment the filter module corresponds to the clarification/filtering column. Both, the inner clarification/filtering column and the binding column can be made of plastic, metal, composite material, glass, or any combination thereof, of any other suitable non-reactive or biocompatible material, able to withstand the force created by a centrifuge being used with the device.

In the preferred embodiment wherein the filter module is in form of a column which can be inserted into a binding column, the inlet or upper open end of the filter module/clarification column is oriented in the same direction as the inlet or upper open end of the binding column, and the outlet of the filter module/clarification column is oriented in the same direction as the outlet of the binding column. The filter module/clarification column can extend all the way through the internal bore so that the outlet of the filter module/clarification column is adjacent to the binding material in the binding column. Alternatively, the filter module/clarification column can extend only partially through the internal bore so that there is a gap between the outlet of the filter module/clarification column and the binding material.

Preferably, the filter module comprises any means for holding the filter module at its intended place when inserted into the binding column, even during centrifugation. Said means may be for example a ring, a crank, a shoulder or similar which preferably either renders the perimeter of the filter module bigger than the perimeter of the binding column, resulting in that the filter module cannot be inserted deeper in the binding column, or it corresponds to a crank or shoulder inside of the binding column, whereon the filter module is placed.

An example of a dual column device wherein both columns having a shoulder is shown in FIG. 1 (the binding material in the outer column is not shown). In said figure the binding column (binding material not shown) is shown as (1), the filter module inserted in the binding column is shown as (2). The elastic filter (3) is placed above the second filter (4).

Further the filter module, e.g. in form of a clarification column, may be designed in a way that it may be inserted into the binding column (without any means for holding the column in place), but can be removed after the filtration step.

If desired, the internal diameter of the binding column can be sized to accommodate the size of the clarification column so that at least a portion of the outer surface of the clarification column contacts a portion of the inner surface of the binding column and provides an close fit between the clarification column and binding column. Such a close fit can be a connection between two surfaces which can be achieved by friction after the surfaces are contacted together. A close fit may also be achieved by shaping the clarification column and binding column so that one or the other (or both) slightly deviate in size from the nominal dimension, or by conical shape of both of the columns. Further, the close fit can be obtained by shaping the crank or shoulder of the columns serving for holding the clarification column in place in a way that the shoulders or cranks are in close contact to each other. The fit between the clarification column and binding column can be sufficiently airtight so that negative pressure applied to the outlet of the binding column will create a vacuum in the dual column system. In general, when positioned into the binding column, the clarification column will be of such a dimension that at least a part of the outer surface of the clarification column contacts at least a part of the inner surface of the binding column and forms a vacuum seal with the binding column when negative pressure is applied. The fit between the outer and clarification column will not prevent the clarification column from being removed from the binding column when negative pressure is not applied.

The binding column comprises a binding material for binding the desired target. The binding material can be any suitable material for capturing the target molecule including, but not limited to, fiber, matrix, resin, membrane, disc or filter, or any other suitable material or combination thereof. The binding material can capture at least one type of target molecule including, but not limited to, nucleic acids. In a preferred embodiment, the binding material is a DNA or RNA binding material, in particular a material able to bind plasmid DNA. Suitable DNA binding materials include silica and non-silica DNA binding materials or a combination thereof. The DNA binding material further can be any suitable chromatography material including, but not limited to, silica gel, aluminium oxide, titanium dioxide, porous glass, polymers, or any combination thereof. Further, the binding material can be a charge switch membrane, including glass fiber or nitrocellulose, or an anion exchange matrix, including derivatized glass fiber. The binding material can be further a combination of several types of said material, e.g. in different layers. The binding material can be located inside of the binding column at or near the outlet. The binding material can be positioned at any suitable location so that the sample passing through the binding column and exiting through the outlet aperture must pass through the binding material.

The binding material preferably comprises pores of a suitable size to provide for adequate flow of the fluid or clarified sample through the device while providing high surface area to which the molecule can bind and thereby providing good yield of the at least one target molecule. The pore size can be any suitable size to allow for the binding of at least one target molecule, e.g. in the range of between approximately 0.5 μm and approximately 5 μm.

The binding column can comprise one or more support elements for the binding material or it may be adhered to the walls of the container, e.g. by a glue, gum, seal or similar adhesive. The support element can maintain the position of the binding material inside of the column. The support element(s) can be of any structure that physically restricts movement of the binding material. Suitable support elements include, but are not limited to, a holder, a ring, a mesh or a frit. Alternatively, the support element can be a modification of the inner surface of the binding column, such as an annular ridge formed on the inner surface of the internal bore.

The present invention further provides a method for clarifying a suspension, comprising at least one target molecule and solid particles, precipitates and/or flocculates, wherein the suspension further may comprise shear-sensitive molecules, including a filtration step involving the filter module as defined above for separating said solid particles, precipitates and/or flocculates from the target molecule, wherein the target molecule remains in solution. In particular said method is suitable for isolating and/or purifying desired target molecules from such a suspension, wherein the suspension further may comprise shear-sensitive molecules, like high molecular weight molecules, including a filtration step involving the filter module as described above. The obtained flow-through contains the desired target molecule in solution essentially free of particulate material like solids, precipitates and flocculates. Further, the obtained solution is preferably essentially free from fragments of shear-sensitive macromolecules or fragments thereof. The flow-through may be contacted with a target binding material to isolate/separate the target from said flow-through.

In a preferred method according to the invention nucleic acids are isolated or purified, preferably DNA, most preferred plasmid DNA.

Preferably, the isolation and/or purification of the nucleic acids includes a step of binding the nucleic acids to a nucleic acid binding material, preferably in a binding column.

In a particular preferred embodiment the present invention provides a method for isolating and/or purifying nucleic acids, comprising the steps of:
(i) contacting a sample comprising nucleic acids with a filter module comprising at least one filter made of an elastic material as described above
(ii) applying an external force to the sample effecting the sample to pass through said filter module

Said method preferably comprises further the steps:
(iii) contacting the flow-through of step (ii) with a nucleic acid binding material
(iv) optionally washing the bound nucleic acids
(v) eluting the nucleic acids from the binding material.

The sample preferably is a cell lysate, particularly a cell lysate comprising genomic DNA and a target biomolecule. The preferred target biomolecule is a target nucleic acid or a protein, in particular plasmid DNA.

The sample containing the desired molecule can be added to a column including the filter module. If the filter module is in the form of a clarification column as described above, the sample is applied in the clarification column through the open end of the clarification column.

Preferably, application of centrifugal force or negative pressure (vacuum) passes the sample through the filter module/clarification column.

Preferably, the flow-through passes into a binding column. As the sample passes through the clarification column, large insoluble molecules can be prevented from passing through the outlet of the clarification column by the comprised filter(s). Due to the elastic properties of the elastic filter, preferably the foam or sponge, shear-sensitive molecules like e.g. genomic DNA are essentially not sheared, even in case high centrifugation speed is applied to the sample. Thus, the amount of fragments of the shear-sensitive molecules is minimized.

The filtered sample comes preferably in contact with the binding material located in the binding column. The binding material can bind the desired molecule or molecules while the centrifugal force or vacuum manipulates the rest of the liquid out of the device through the outlet of the binding column. The filter module/clarification column can then be removed. One or more washing solutions may be optionally added and forced by centrifugation or vacuum to pass through the binding material and out of the device. The washing increases purification of the desired molecules by removing unbound molecules, impurities, or other debris from the binding material. An eluting solution can then added to the binding column to elute the desired molecule from the binding material. The eluate preferably is collected. Multiple aliquots of eluting solution can be used.

In a preferred embodiment of the method, the suspension or liquid mixture is a cell lysate and the filter module operates in form of a clarification column. The clarified lysate is passed through the binding material which binds a nucleic acid of interest, preferably plasmid DNA. The clarification column can then be removed and the bound plasmid DNA is eluted and collected. The dual column device of the present invention enables clarification and binding in a single short (1-3 minute) centrifugation or vacuum step.

In a particular preferred embodiment the present invention provides a method for isolating and/or purifying plasmid DNA, comprising the steps of:
(i) contacting a cell lysate comprising genomic DNA and plasmid DNA with a filter module comprising at least one filter made of a self-supporting foam or sponge
(ii) applying an external force to the sample effecting the sample to pass through said filter module
(iii) contacting the flow-through of step (ii) with a DNA binding material
(iv) optionally washing the bound DNA
(v) eluting the DNA from the binding material.

Further provided herein are kits for isolating a target molecule of interest from a sample. A kit for isolating a target molecule of interest comprises at least a filter module as described herein.

Preferably, the kit comprises a clarification/binding device for isolation of at least one target molecule from a sample comprising: a filter module/clarification column configured to receive the sample, the clarification column comprising at least one filter made of an elastic filter material as described above, preferably a self-supporting foam or sponge, configured to filter at least one non-target molecule from the sample, and a binding column configured to receive the filtered sample from the filter module/clarification column, the binding column comprising a binding material for binding at least one target molecule.

In an alternative preferred embodiment the kit comprises a column comprising a filter module including at least one filter made of an elastic material as described above, preferably a foam or sponge, and a binding material, wherein preferably the filter module is removable from the column.

The kit may further comprise at least one further ingredient, selected from: at least one lysis buffer; at least one RNase stock solution; at least one resuspension buffer; at least one neutralization buffer; at least one wash buffer; at least one elution buffer; instructions for carrying out the target isolation/purification method.

### FIGURES

FIG. 1 shows a filter module in a spin column for combined filtration and binding, wherein the binding material in the spin column is not shown. (1) Spin column (binding material not shown, (2) Filter module, (3) Soft, elastic upper filter material, (4) compact second filter.

FIG. 2 shows a possible design of the filter module, wherein the bottom of the column represents a supporting means (filter materials are not shown)

FIG. 3 shows plasmid preparations as isolated according to Example 2 on an agarose gel.

1 filter with non binding silica as lower filter material and a porous frit, PE, 10 μm as upper filter material
2 filter with a porous frit PE, 7-12 μm as lower filter material and a Gaze PET, 20 μm as upper filter material
3 filter with non binding silica as lower filter material and a porous frit, PE, 20-60 μm as upper filter material
4 filter with non binding silica as lower filter material and a porous frit, PE, 10-20 μm as upper filter material
(5) reference: 10 minutes centrifugation for pelleting the precipitate instead of filtering

FIG. 4 shows mini preparations on a agarose gel including (1) a needle punched felt and the (2) reference: 10 minutes centrifugation for pelleting the precipitate according to Example 3.

FIG. 5 shows an agarose gel representing the results of plasmid preparations performed according to Example 1, using a filter module comprising the foam (1) and of preparations according to the reference (10 minutes centrifugation for pelleting the precipitate (2)

FIG. 6 shows the content of genomic DNA in comparison to the isolated plasmid according to Example 5, dependent from the type of filter used for lysate clearance. Mini preparations are shown as follows. (1) reference: 10 minutes centrifugation for pelleting the precipitate instead of filtering, (2) the plasmid isolated using a foam as filtering material and (3) the plasmid isolated using a needle punched felt as filtering material.

FIG. 7 shows the content of genomic DNA in comparison to the isolated plasmid according to Example 5, dependent from the type of filter used for lysate clearance. Two mini preparations are shown. (1) reference: 10 minutes centrifugation for pelleting the precipitate (2) the plasmid isolated using a foam as filtering material and (3) the plasmid isolated using a needle punched felt as filtering material.

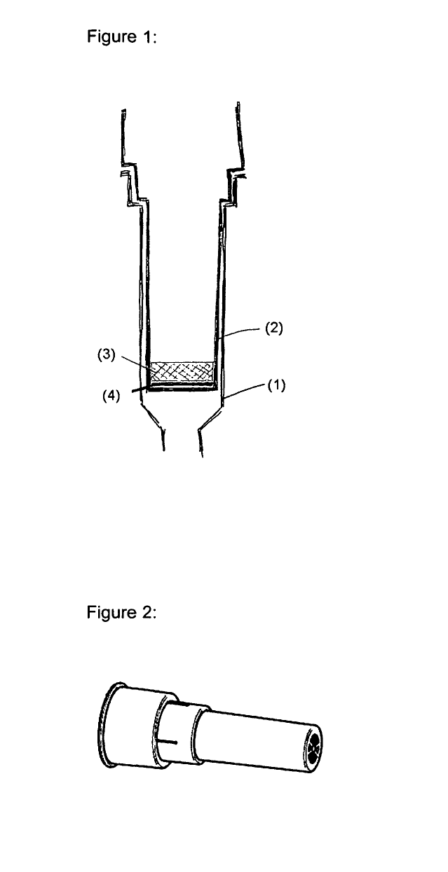

### EXAMPLES

### Example 1

According to the following protocols plasmid DNA was isolated from bacterial cells. Cells were harvested from bacterial cell culture by centrifugation by pelleting the cells in a 1.5 ml Eppendorf cup. The buffers, solutions and DNA binding columns used are those from the commercially available QIAprep Kit (QIAGEN; Hilden, Germany), designed for plasmid DNA isolation.

### Protocol Centrifuge

All centrifugation steps are carried out at 13,000 rpm.
1. The pelleted bacterial cells were resuspended in 250 μl Buffer P1 and transferred to a microcentrifuge tube.
2. 250 μl Buffer P2 were added and mixed thoroughly by inverting the tube 4-6 times.
3. 350 μl Buffer N3 were added and mixed immediately and thoroughly by inverting the tube 4-6 times.
4. The whole sample volume including precipitates from step 4 was applied to a filter module in form of a column, inserted in a QIAprep Spin Column by decanting or pipetting. The filter module comprised a polyurethane foam and a non-binding silica membrane (i.e. a silica membrane not binding nucleic acids).
5. The whole assembly was centrifuged for 1 min, the flow-through was discarded.
6. The filter module was removed from QIAprep spin column and discarded.
7. The QIAprep spin column was washed by adding 0.5 ml Buffer PB and centrifuging for 30-60 s. Flow-through was discarded.
8. The QIAprep spin column was washed by adding 0.75 ml Buffer PE and centrifuging for 30-60 s.
9. The flow-through was discarded, and the column was centrifuged for an additional 1 min to remove residual wash buffer.
10. The QIAprep column was placed in a clean 1.5 ml microcentrifuge tube. To elute DNA, 50 μl Buffer EB (10 mM Tris.Cl, pH 8.5) were added to the center of each QIAprep spin column, let stand for 1 min, and centrifuged for 1 min. The eluate was collected.

### Protocol Vacuum

1. A vacuum manifold and QIAprep spin columns were prepared according to the details in QIAprep Miniprep handbook.
2. The pelleted bacterial cells were resuspended in 250 μl Buffer P1 and transferred to a microcentrifuge tube.
3. 250 μl Buffer P2 were added and mixed thoroughly by inverting the tube 4-6 times.
4. 350 μl Buffer N3 were added and mixed immediately and thoroughly by inverting the tube 4-6 times.
5. The whole sample volume including precipitates from step 4 was applied to a filter module in form of a column, inserted in a QIAprep Spin Column which was on the vacuum manifold by decanting or pipetting. The filter module comprised a polyurethane foam and a non-binding silica membrane.
6. The vacuum source was switched on to draw the solution through the Filter module and the QIAprep spin columns, then vacuum source was switched off.
7. The filter module was removed from QIAprep spin column and discarded.
8. The QIAprep spin column was washed by adding 0.5 ml Buffer PB. Vacuum source was switched on to draw the wash solution through the column, and then vacuum source was switched off.
9. The QIAprep spin column was washed by adding 0.75 ml Buffer PE. Vacuum source was switched on to draw the wash solution through the column, and then vacuum source was switched off.
10. The QIAprep spin columns were placed into a 2 ml collection tube and transferred to a microcentrifuge tube. Centrifugation for 1 min at 13,000 rpm to remove residual wash buffer was carried out.
11. The QIAprep column was placed in a clean 1.5 ml microcentrifuge tube. To elute DNA, 50 μl Buffer EB (10 mM Tris.Cl, pH 8.5) were added to the center of each QIAprep spin column, let stand for 1 min, and centrifuged for 1 min at 13,000 rpm. The eluate was collected.
   Protocol for Reference as described in the QIAprep Miniprep Handbook (June 2005) on p 22-23
1. The pelleted bacterial cells were resuspended in 250 μl Buffer P1 and transferred to a microcentrifuge tube.
2. 250 μl Buffer P2 were added and mixed thoroughly by inverting the tube 4-6 times.
3. 350 μl Buffer N3 were added and mixed immediately and thoroughly by inverting the tube 4-6 times.
4. The sample was centrifuged for 10 min at 13,000 rpm in a table top microcentrifuge.
5. The supernatant from step 4 was transferred to a QIAprep spin column.
6. The QIAprep spin column was centrifuged for 60 s. The flow through was discarded.
7. The QIAprep spin column was washed by adding 0.5 ml Buffer PB and centrifuging for 30-60 s. Flow-through was discarded.
8. The QIAprep spin column was washed by adding 0.75 ml Buffer PE and centrifuging for 30-60 s.
9. The flow-through was discarded, and the column was centrifuged for an additional 1 min to remove residual wash buffer.
10. The QIAprep column was placed in a clean 1.5 ml microcentrifuge tube. To elute DNA, 50 μl Buffer EB (10 mM Tris.Cl, pH 8.5) or water was added to the center of each QIAprep spin column, let stand for 1 min, and centrifuged for 1 min. The eluate was collected.

As a “reference” samples were treated by the same methods, respectively, with the exception that instead of the filtering step the solids, precipitates and flocculates are removed by pelleting via centrifugation (10 min 13,000 rpm) and the supernatant is transferred into a new tube and further processed.

### Example 2

Comparison of plasmid mini preparations performed using filtration (filter module containing a rigid upper filter material) for lysate clearing or centrifugation (pelletation) as reference:

Plasmid pUC19 was isolated from 5 ml LB culture of E. coli TOP10F cells according to the protocol for centrifugation as described in Example 1. Four different types of filter materials were used as the upper filter in the filter module.

1 a filter with non binding silica as lower filter material and a porous frit, PE, 10 μm as upper filter material,
2 a filter with a porous frit PE, 7-12 μm as lower filter material and a Gaze PET, 20 μm as upper filter material,
3 a filter with non binding silica as lower filter material and a porous frit, PE, 20-60 μm as upper filter material,
4 a filter with non binding silica as lower filter material and a porous frit, PE, 10-20 μm as upper filter material.

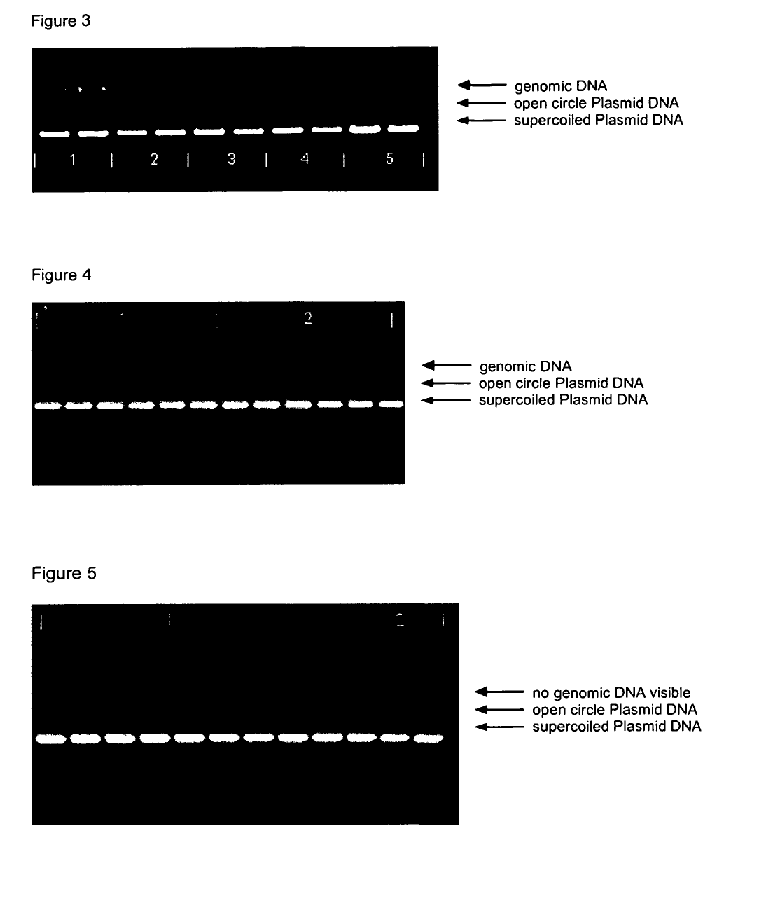

All samples were analyzed on an agarose gel. The gel is shown in FIG. 3. 150 ng DNA according to OD₂₆₀ of each of the eluates comprising the respective plasmid DNA were run on an agarose gel. As can be seen, all the sintered materials (1-4) effect a clearly higher gDNA contamination of the desired plasmid DNA than precipitation by centrifugation of the crude lysate (5).

### Example 3

### Comparison of Filtration with Rigid Upper Filter Material and Pelletation as Reference: FIG. 4

Plasmid pUC19 was isolated from 5 ml LB culture of E. coli DH10B cells or plasmid pCMVβ was isolated from 5 ml LB culture of E. coli DH5alpha cells, respectively, according to the protocol for centrifugation as described in Example 1. As upper filter material in the filter module, needle punched felt (1) (a rigid filter material) was used in comparison to the reference (2). All samples were analyzed in triplicate. 150 ng DNA according to OD₂₆₀ of each of the eluates comprising the respective plasmid DNA were run on an agarose gel. The gel showed that the samples filtered with needle punched felt include clearly more (fragmented) genomic DNA than the samples cleared by pelletation.

### Example 4

### Comparison of Filtration with Elastic/Soft Upper Filter Material and Pelletation as Reference: FIG. 5

Plasmid pUC19 was isolated from 5 ml LB culture of E. coli DH10B cells or plasmid pCMVβ was isolated from 5 ml LB culture of E. coli DH5alpha cells, respectively, according to the protocol for centrifugation as described in Example 1. As upper filter in the filter module, polyurethane, polyethylene or polystyrene foam was used in comparison to the reference pelletation of the precipitate. All samples were analyzed in triplicate. 150 ng DNA according to OD₂₆₀ of each of the eluates comprising the respective plasmid DNA were run on an agarose gel. According to the gel analysis as shown in FIG. 5, there is no genomic DNA visible for both types of samples.

### Example 5

### Quantification of Genomic DNA Contamination

To quantify the difference of genomic DNA contamination in the samples again plasmids were isolated according to the protocol of Example 1, which were pUC19 from 5 ml LB culture of TOP10F cells and pBRCMVβ from 5 ml LB culture of DH5α cells. As upper filter material either a polyurethane foam as in Example 4 or a needle punched felt as in Example 3 was used. After elution of the plasmid DNA 125 ng plasmid DNA were used as a template in a real time PCR. To determine the amount of genomic DNA contamination primers annealing to a chromosomal pyruvat kinase gene and a DNA probe were used. The sequence of the primers and the probe were as follows:

| | |
|---|---|
| primer A | Tcg taa gcg ttc tga cgt tat c |
| primer B | Cat gat gcc gtc aga ggc ttc gag |
| probe | FAM-acc tga aag cgc acg gcg gcg aa |

The amount of contaminating genomic DNA was quantified by means of a standard series of genomic DNA with known amounts.

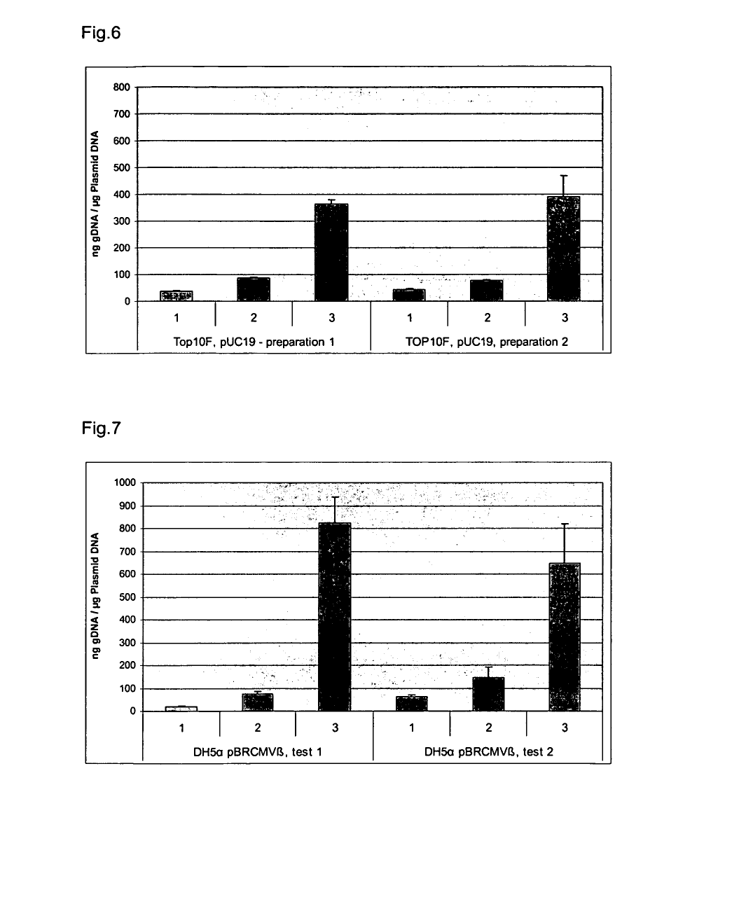

As shown in FIGS. 6 & 7 contamination of genomic DNA in the samples filtered with the rigid material was much higher than in the samples filtered with the foam according to the invention. As a reference plasmid preparations using pelletation of the precipitate in the lysate were used as a template. In FIGS. 6 & 7 respective samples (1) represent the reference: 10 minutes centrifugation for pelleting the precipitate, samples (2) the plasmid isolated using a filter module containing the foam as upper filter material and non binding silica as lower filter and samples (3) the plasmid isolated using a filter module comprising the needle punched felt (a more rigid material) as upper filter material and non binding silica as lower filter.

1. A clarification/binding device comprising a filter module, wherein said filter module comprises an elastic filter material.

2. Use of a filter module comprising an elastic filter material in a method for isolating nucleic acids.

3. The clarification/binding device according to claim 1 or use according to claim 2, wherein the elastic filter material is an open-cell foam or a sponge.

4. The clarification/binding device or use according to claim 3, wherein the filter is made of a foamed polyethylene, polypropylene, polyurethane, polyester, polyether, polystyrene, melamine, natural sponges, animal fiber sponges or plant fiber sponges.

5. The clarification/binding device or use according to any of claims 1 to 4, wherein the filter module further comprises a second filter, made of a fiber material.

6. The clarification/binding device or use according to any of claims 1 to 5, wherein the second filter is placed below or behind the elastic filter in flow direction and the pore size of the second filter is smaller than the pore size of the elastic filter.

7. The clarification/binding device or use according of any of claims 1 to 6, wherein the device is a single column clarification/binding device, containing the filter module in a column further comprising a target binding material, or the device is dual column clarification/binding device, wherein the filter module is present in form of a further column which is inserted in the binding column.

8. The clarification/binding device or use according of any of claims 1 to 7, wherein the filter module is removable from the binding column.

9. Method for clarifying a suspension, comprising at least one target molecule and solid particles, precipitates and/or flocculates, wherein the suspension further optionally may comprise shear-sensitive molecules, including a filtration step involving the filter module as defined in any of claims 1 to 8 for separating said solid particles, precipitates and/or flocculates from the target molecule, wherein the target molecule remains in solution.

10. Method according to claim 9 for isolating and/or purifying nucleic acids as the target molecule, comprising the steps of:
(i) contacting a sample suspension comprising nucleic acids with a filter module as defined in any of claims 1 to 8
(ii) applying an external force to the sample effecting the sample to pass through said filter module.

11. The method of claim 10, comprising further the steps:
(iii) contacting the flow-through of step (ii) with a nucleic acid binding material
(iv) optionally washing the bound nucleic acids
(v) eluting the nucleic acids from the binding material.

12. The method of any of claims 9 to 11, wherein the target molecule is plasmid DNA or RNA

13. The method of any of claims 9 to 12, wherein external force applied in step (ii) is centrifugal force or vacuum.

14. The method of any of claims 11 to 13, wherein the flow-through in step (iii) is contacted with a nucleic acid binding material by using a dual-column device.

15. Kit for carrying out the method of any of claims 9 to 14, comprising a filter module or a clarification/binding device according to any of claims 1 to 8 and optionally any further ingredient, selected from: at least one lysis buffer; at least one RNase stock solution; at least one resuspension buffer; at least one neutralization buffer; at least one wash buffer; at least one elution buffer; and/or instructions for carrying out the target isolation/purification method.
